(19) United States
(12) Patent Application Publication
(10) Pub. No.: US 2026/0294669 A1
(43) Pub. Date: Oct. 1, 2026

(54) METHOD AND APPARATUS FOR A UROSTOMY UTILITY POUCH

(71) Applicant: Rennie Terbogt, Toronto (CA)

(72) Inventor: Rennie Terbogt, Toronto (CA)

(21) Appl. No.: 19/089,084

(22) Filed: Mar. 25, 2025

(51) Int. Cl. A61F 5/44

(52) U.S. Cl. CPC A61F 5/4401; A61F 5/4404

(57) ABSTRACT

A urostomy utility pouch to simultaneously wick and stabilize bodily fluid. The urostomy utility pouch includes a main body having an exterior surface exposed to a surrounding environment, an interior surface opposite to the exterior surface and defining a chamber, and an opening defined in the main body providing access to the chamber. The urostomy utility pouch includes a connector that operably engages with the main body inside of the opening and adapted to couple with a skin patch positioned about a patient's stoma. The urostomy utility pouch includes an absorber that operably engages with the interior surface of the main body inside of the chamber and is configured to wick and stabilize bodily fluid from the patient's stoma while the main body is configured to hold the bodily fluid inside of the chamber.

### TECHNICAL FIELD

This disclosure is directed to a urostomy utility pouch configured to collect and store bodily fluid excreted from a patient's stoma.

### BACKGROUND ART

Subsequent to performing a cystectomy (urinary bladder removal) on a patient, a physician must perform a urinary diversion in order for bodily fluid (such as urine) to travel from the patient's kidneys to the outside of the patient for removal purposes. During this urinary diversion process, a urostomy surgery is performed to create an opening (or stoma) in the patient's abdomen to allow urine, or other bodily fluid waste to be removed from the patient's body. With respect to a urostomy surgery, a stoma is created in the patient's abdomen and the patient's urinary tract is attached to the stoma to allow urine and similar bodily fluid waste to leave the patient's body.

Once a stoma is placed, the patient must wear a urostomy pouch or bag that is used to collect urine and other bodily fluid waste from the urinary tract for disposal purposes. Normally, the urostomy pouch is secured to the patient's body and surrounds the patient's stoma to collect the bodily fluid by using a skin patch or similar adhering component. Once adhered, the urostomy pouch is secured to the skin patch in order for the urostomy pouch to collect and store bodily fluid that excretes from the patient's stoma. The urostomy pouch provides assistance to the patient in multiple ways, including the act of managing urinary function and urinary output, improving quality of life, and preventing infections or other health complications.

While such use of these urostomy pouches are helpful in collecting urine, common urostomy pouches have major disadvantages to patients. In one instance, common urostomy pouches are generally long in length which means that, depending on the overall stature the patient, these urostomy pouches hang or suspend below the patients'hips and into the patients'groins which may create a lifestyle nuisance as well as the fear of the urostomy pouch being grabbed or hooked by exterior elements that surround the patient. Particularly, sloshing of the bodily fluid stored inside of common urostomy pouches may occur even in the lightest or less intense sporting activities such as tennis, pickleball, curling, cycling, etc. In another instance, the sealing capabilities of common urostomy pouches lack depending on the position of the patients. As such, patients that are oriented in horizontal or lying down positions may be at a risk due to gravity failing to assist in directing the bodily fluid to the urostomy pouches which may create leaks or escapements of such bodily fluid before reaching the urostomy pouches. Continuing with the horizontal positioning of patients, bodily fluid that is stored in these common urostomy pouches may escape and back-up into the patient's urinary tract when gravity is unable to assist in these positions. With such disadvantage, patients may be more prone to infections resulting from the bodily fluid backing up into the patient's body.

### SUMMARY OF THE INVENTION

The presently disclosed urostomy pouches provided herein include an absorber for immediately wicking and stabilizing bodily fluid from a respective stoma provided on a patient. Such use of the absorber allows patients to be provided in any suitable position or orientation (e.g., in a vertical or standing position, a horizontal or lying down position, or at angle between the vertical and horizontal positions). The stabilizing technology of the absorber also transitions the bodily fluid from a liquid state to a solid-like state to prevent sloshing and movement of the liquid inside of the pouch when the patient is performing physical activities. The presently disclosed urostomy pouches provided herein also provide discrete and low-profile designs so that such urostomy pouches are concealed underneath clothing as well as concealable during physical or sporting activities. The presently disclosed urostomy pouches provided herein also include a connector that allows fluid to be directed at the absorber in two separate drainage pathways. Particularly, the connector includes a drainage passageway that provides a first drainage pathway and a plurality of drainage ports that provides a second drainage pathway.

In one aspect, an exemplary embodiment of the present disclosure may provide a urostomy utility pouch. The urostomy utility pouch includes a main body having an exterior surface exposed to a surrounding environment and an interior surface opposite to the exterior surface and defining a chamber; an opening defined in the main body providing access to the chamber; a connector operably engaged with the main body inside of the opening and adapted to couple with a skin patch positioned about a patient's stoma; and an absorber operably engaged with the interior surface of the main body inside of the chamber; wherein the absorber is configured to wick and stabilize bodily fluid from the patient's stoma while the main body is configured to hold the bodily fluid inside of the chamber.

This exemplary embodiment or another exemplary embodiment may further include that the pouch is configured to be positioned entirely above patient's hips and remote from a patient's groin. This exemplary embodiment or another exemplary embodiment may further include that the connector comprises: an engagement end adapted to engage with an attachment clip of the skin patch; a chamber end opposite to the engagement end and positioned inside of the chamber; and a drainage passageway defined between the engagement end and the chamber end that is configured to collect and guide the bodily fluid from the patient's stoma to a first absorbing area of the absorber and the chamber. This exemplary embodiment or another exemplary embodiment may further include that the absorber engages with the chamber end and is positioned outside of the drainage passageway. This exemplary embodiment or another exemplary embodiment may further include that the connector further comprises: at least one drainage port extending between an inner surface of the connector and an outer surface of the connector and being in fluid communication with the drainage passageway; wherein the at least one drainage port is configured to drain the bodily fluid from the drainage passageway to a second absorbing area of the absorber and the chamber. This exemplary embodiment or another exemplary embodiment may further include a first drainage pathway defined along a length of the drainage passageway of the connector; and a second drainage pathway defined along a length of the at least one drainage port of the connector that is non-parallel to the first drainage pathway. This exemplary embodiment or another exemplary embodiment may further include that the connector further comprises: an interlocking groove extending from the engagement end to a base wall of the connector; wherein the interlocking groove is adapted to interlockingly engage with an attachment clip of the skin patch. This exemplary embodiment or another exemplary embodiment may further include that the at least one drainage port and the interlocking groove are free from being in fluid communication with one another along the inner surface of the connector. This exemplary embodiment or another exemplary embodiment may further include that the connector further comprises: a plurality of drainage ports extending between an inner surface of the connector and an outer surface of the connector and being in fluid communication with the drainage passageway; wherein each drainage port of the plurality of drainage ports is configured to drain the bodily fluid from the drainage passageway to the chamber. This exemplary embodiment or another exemplary embodiment may further include a cap removably engageable with the connector to prevent the bodily fluid from escaping the chamber; wherein the cap is engaged with the connector when the connector is decoupled from the skin patch. In this exemplary embodiment, the cap may secondarily be used to seal the urostomy bag previously in use when the exemplary pouch is attached to the skin patch. This exemplary embodiment or another exemplary embodiment may further include that the connector further comprises: at least one pull tab operably engaged with an outer surface of the connector. This exemplary embodiment or another exemplary embodiment may further include that the main body is made of biodegradable material.

In another aspect, an exemplary embodiment of the present disclosure may provide a method of storing bodily fluid excreted from a patient's stoma. The method includes steps of: coupling a connector of a urostomy utility pouch with a corresponding skin patch surrounding the patient's stoma; capturing the bodily fluid with the connector; wicking the bodily fluid from the connector by an absorber of the urostomy utility pouch inside of a main body of the urostomy utility pouch; and stabilizing the bodily fluid excreted from the patient's stoma by the absorber.

This exemplary embodiment or another exemplary embodiment may further include a step of positioning the urostomy utility pouch entirely above patient's hips and remote from a patient's groin. This exemplary embodiment or another exemplary embodiment may further include that the steps of directing the bodily fluid and wicking the bodily fluid further comprises: directing the bodily fluid into the chamber and the absorber through a drainage passageway defined in the connector; and wicking the bodily fluid from the connector by the absorber at a first absorbing area. This exemplary embodiment or another exemplary embodiment may further include the steps of directing the bodily fluid and wicking the bodily fluid further comprises: directing the bodily fluid from the drainage passageway and into the chamber and the absorber through at least one drainage port defined in the connector; and wicking the bodily fluid from the connector by the absorber at a second absorbing area that is substantially orthogonal to the first absorbing area of the absorber. This exemplary embodiment or another exemplary embodiment may further include that the step of coupling the connector of the urostomy utility pouch with the corresponding skin patch further comprises: engaging an attachment clip of the skin patch with an interlocking groove defined in the connector; and wherein the steps of directing the bodily fluid and wicking the bodily fluid further comprises: directing the bodily fluid into the chamber and the absorber through at least one drainage port defined in the connector; wherein the interlocking groove and the at least one drainage port are free from being in fluid communication with one another. This exemplary embodiment or another exemplary embodiment may further include steps of removing the urostomy utility pouch from the skin patch; engaging a cap with the connector; and sealing the chamber of the urostomy utility pouch with the cap at the opening. This exemplary embodiment or another exemplary embodiment may further include that a main body of the urostomy utility pouch is made of biodegradable material.

In yet another aspect, an exemplary embodiment of the present disclosure may provide a urostomy utility kit. The urostomy utility kit also includes a urostomy utility pouch removably coupled with a skin patch, the urostomy utility pouch comprising: a main body having an exterior surface exposed to a surrounding environment and an interior surface opposite to the exterior surface and defining a chamber; an opening defined in the main body providing access to the chamber; a connector operably engaged with the main body inside of the opening and adapted to couple with the skin patch positioned about the patient's stoma; and an absorber operably engaged with the interior surface of the main body inside of the chamber; wherein the absorber is configured to wick bodily fluid from the patient's stoma while the main body is configured to hold the bodily fluid inside of the chamber. The urostomy utility kit also includes a cap removably engageable with the urostomy utility pouch to prevent the bodily fluid from escaping the chamber. The urostomy utility kit may include the skin patch that is adapted to adhere to a patient's skin and to surround the patient's stoma.

### BRIEF DESCRIPTION OF THE DRAWINGS

One or more exemplary embodiment(s) of the present disclosure is set forth in the following description, is shown in the drawings and is particularly and distinctly pointed out and set forth in the appended claims. The accompanying drawings, which are incorporated in and constitute a part of the specification, illustrate various example configurations and methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1A (FIG. 1A) is a front elevation view of a PRIOR ART urostomy utility kit secured to a patient.

FIG. 1B (FIG. 1B) is a partial side elevation view of PRIOR ART urostomy utility kit shown in FIG. 1 taken in the direction of line 1B-1B in FIG. 1A.

FIG. 1C (FIG. 1C) is an exploded side elevation view of PRIOR ART urostomy utility kit shown in FIG. 1 positioned away from a patient's stoma.

FIG. 1D (FIG. 1D) is a rear elevation view of a urostomy utility pouch of the PRIOR ART urostomy utility kit shown in FIG. 1A.

FIG. 1E (FIG. 1E) is a rear elevation view of a urostomy skin patch of the PRIOR ART urostomy utility kit shown in FIG. 1A coupled to the urostomy utility pouch.

FIG. 2 (FIG. 2) is a front elevation view of a urostomy utility kit being used by a patient in accordance with a first aspect of the present disclosure.

FIG. 3 (FIG. 3) is a partial side elevation view of the urostomy utility kit shown in FIG. 2 taken in the direction of line 3-3 in FIG. 2.

FIG. 4 (FIG. 4) is an exploded view of the urostomy utility kit shown in FIG. 2 positioned away from a patient's stoma.

FIG. 5 (FIG. 5) is an exploded view of the urostomy utility kit shown in FIG. 2, wherein the urostomy utility kit includes a urostomy utility pouch and a skin patch.

FIG. 6 (FIG. 6 ) is a rear elevation view of the urostomy utility pouch shown in FIG. 2.

FIG. 7 (FIG. 7) is a cross-sectional view of the urostomy utility pouch taken in the direction of line 7-7 in FIG. 6.

FIG. 7A (FIG. 7A) is an enlargement of the highlighted region denoted in FIG. 7.

FIG. 8A (FIG. 8A) is an operational view of introducing the skin patch proximate to a patient's stoma.

FIG. 8B (FIG. 8B) is another operational view continuing from FIG. 8A, wherein the skin patch is secured proximate to a patient's stoma and the urostomy utility pouch is being introduced to the skin patch and the patient's stoma.

FIG. 8C (FIG. 8C) is another operational view continuing from FIG. 8B, wherein the urostomy utility pouch is coupled to the skin patch.

FIG. 9 (FIG. 9) is a sectional view of the skin patch and the urostomy utility pouch secured to the patient, wherein the patient's stoma is housed inside of the urostomy utility pouch.

FIG. 9A (FIG. 9A) is an enlargement of the highlighted region denoted in FIG. 9.

FIG. 10 (FIG. 10) is an operational view continuing from FIG. 8C, wherein the bodily fluid that is excreted from the patient's stoma is wicked into an absorber of the urostomy utility pouch and collected inside of a main body of the urostomy utility pouch.

FIG. 11 (FIG. 11) is an operational view continuing from FIG. 10, wherein the urostomy utility pouch is decoupled from the skin patch and discarded with collected bodily fluid.

FIG. 12 (FIG. 12) is a rear perspective view of a connector of a urostomy utility pouch in accordance with a second aspect of the present disclosure.

FIG. 13 (FIG. 13) is a front perspective view of the connector shown in FIG. 12.

FIG. 14 (FIG. 14) is a sectional view of the connector provided in FIG. 12 taken in direction of line 14-14 shown in FIG. 12.

FIG. 15 (FIG. 15) is another sectional view of the connector provided in FIG. 12 taken in direction of line 15-15 shown in FIG. 13.

FIG. 16 (FIG. 16) is an exploded view an urostomy utility kit in accordance with the second aspect of the present disclosure, wherein the urostomy utility kit includes a urostomy utility pouch having the connector shown in FIG. 12, a skin patch, and a sealer cap.

FIG. 17 (FIG. 17) is a sectional view of the urostomy utility pouch provided in FIG. 16 being coupled to the skin patch and secured about a patient's stoma; wherein the connector of the urostomy utility pouch provides two drainage passageways for bodily fluid excreted from a patient's stoma.

FIG. 18 (FIG. 18) is a front elevation view of a urostomy utility pouch in accordance with a third aspect of the present disclosure.

FIG. 19 (FIG. 19) is a diagrammatic flowchart for a method of storing bodily fluid from a patient's stoma.

Similar numbers refer to similar parts throughout the drawings.

### DETAILED DESCRIPTION

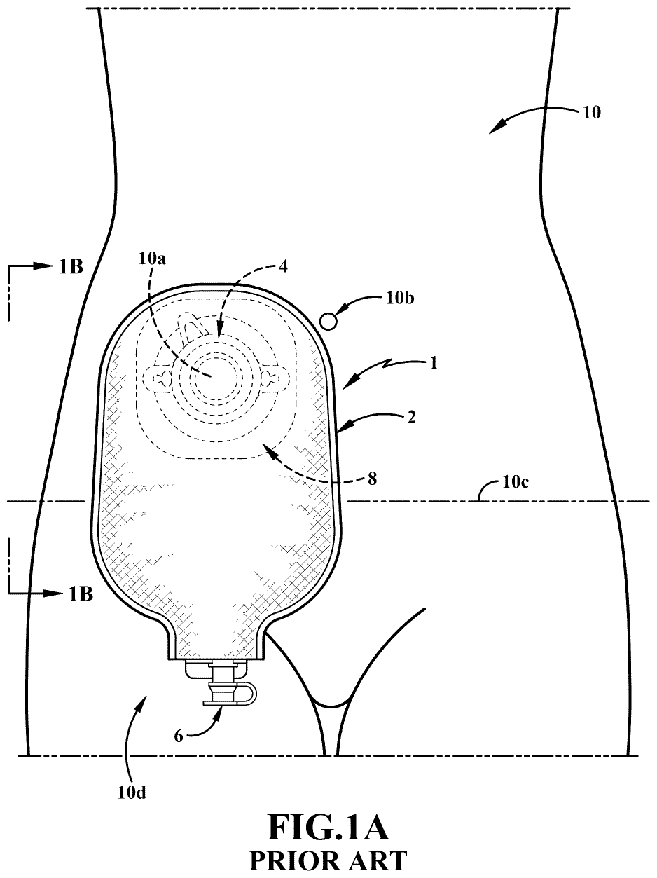

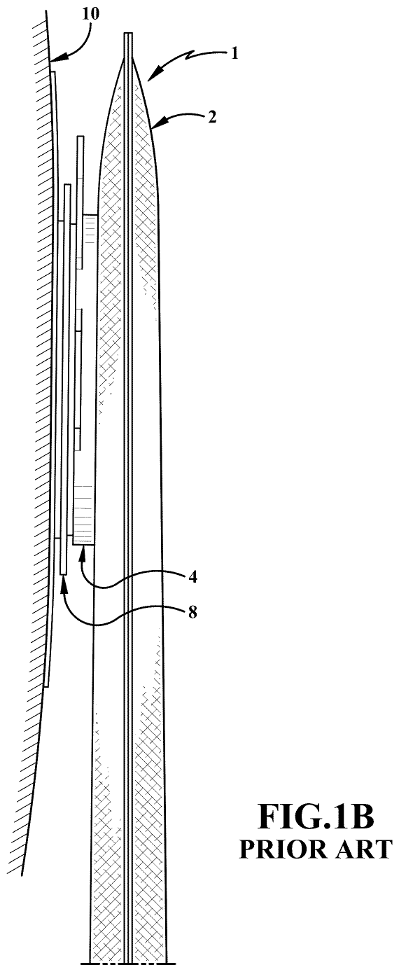

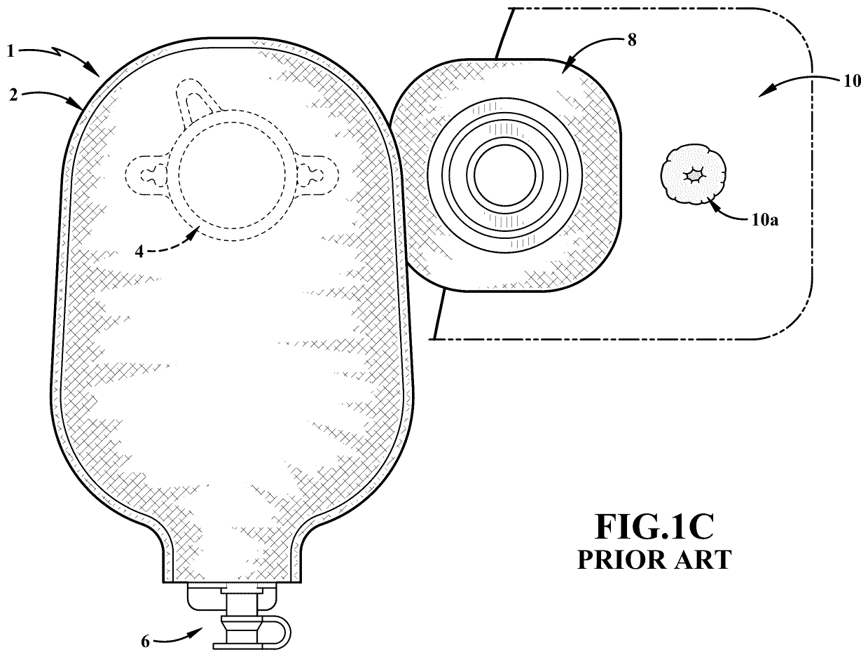

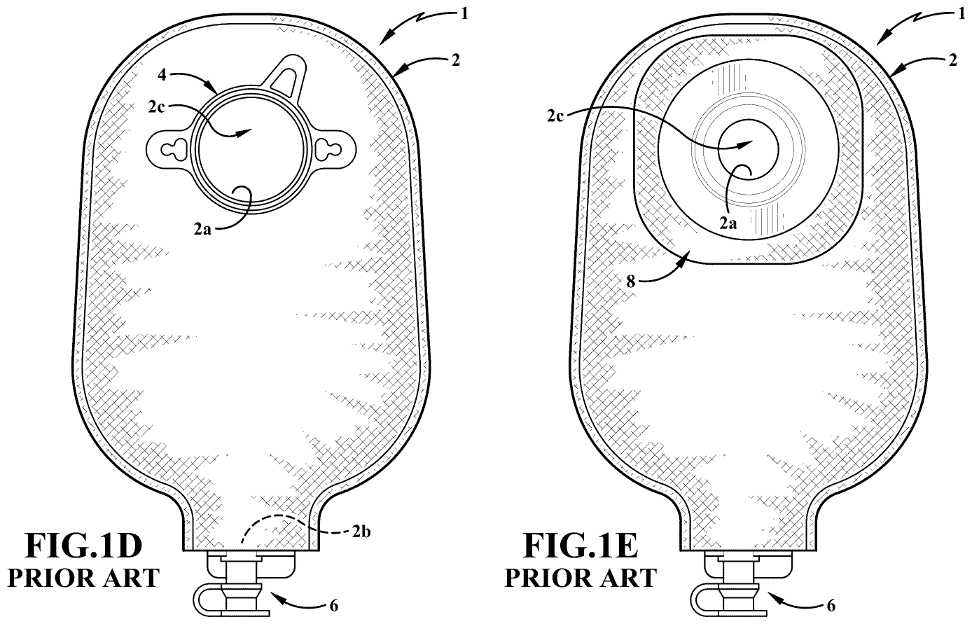

FIGS. 1A-1E illustrate a PRIOR ART urostomy bag kit that includes a PRIOR ART urostomy bag or pouch (hereinafter “PRIOR ART bag”) generally referred to as 1 for collecting bodily fluid from a patient's stoma. In FIG. 1D, PRIOR ART bag 1 includes a main body 2 that defines an inlet opening 2a, an outlet opening 2b that is spaced apart from the inlet opening 2a and positioned vertically below outlet opening 2b, and a chamber 2c defined by main body 2 that is accessible from inlet opening 2a and outlet opening 2b. Still referring to FIG. 1D, PRIOR ART bag 1 also includes a connector 4 that is operably engaged with main body 2 at inlet opening 2a. In the PRIOR ART embodiment, connector 4 is configured to receive and direct bodily fluid excreted from a patient's stoma into the chamber 2c of main body 2. Still referring to FIG. 1D, PRIOR ART bag 1 also includes a drain 6 that is operably engaged with main body 2 at outlet opening 2b. In the PRIOR ART embodiment, the drain 6 may be provided in an open position to empty the main body 2 of bodily fluid when main body 2 reaches a full or filled stated and be provided in a closed position to collect and retain bodily fluid inside of main body 2.

PRIOR ART urostomy bag kit also includes a PRIOR ART skin patch 8. In the PRIOR ART embodiment, PRIOR ART skin patch 8 is configured to adhere and/or secure to a patient 10 that surrounds a patient's stoma 10a (see FIG. 1C). The PRIOR ART skin patch 8 may include an attachment clip or arm that interlockingly engages the connector 4 of PRIOR ART bag 1 and skin patch 8 with one another. Upon engagement, the PRIOR ART bag 1 is configured to hang or suspend from the PRIOR ART skin patch 8 so that bodily fluid excreted from the patient's stoma 10a is directed downwardly from PRIOR ART skin patch 8 to the chamber of main body 2 of PRIOR ART bag 1 by gravity.

While such PRIOR ART urostomy bag kit is feasible in collecting bodily fluid excreted from patient's stoma 10a in limited arrangements, the PRIOR ART bag 1 of PRIOR ART urostomy bag kit creates a lifestyle nuisance to the patient given the length and cumbersomeness of the PRIOR ART bag 1. In most instances with PRIOR ART bag 1, PRIOR ART bag 1 extends past patient's hips (denoted with a dot-dot-dashed line labeled 10c in FIG. 1A) and near a patient's groin 10d which may create an unwanted nuisance in everyday life for a patient wearing said PRIOR ART bag 1. Additionally, PRIOR ART bag 1 creates a hygiene risk to the patient wearing PRIOR ART bag 1 since PRIOR ART bag 1 must be emptied based on the structural design of PRIOR ART bag 1. Since the bodily fluid stored inside of PRIOR ART bag 1 is biohazardous, the patient is consistently at risk of health issue each time the patient drains and empties the PRIOR ART bag 1.

Furthermore, the collection of bodily fluid by PRIOR ART bag 1 from the patient's stoma 10a is merely achieved through gravity which, at times, may be insufficient when small or minimal amounts of bodily fluid are excreted from the patient's stoma 10a causing leaks or escapement bodily fluid from PRIOR ART bag 1. Additionally, such leaks or escapement bodily fluid from PRIOR ART bag 1 may occur when the patient is arranged in a horizontal position (i.e., lying down on furniture or sleeping). In these instances, PRIOR ART bag 1 is unable to capture most or all of the bodily fluid excreted from the patient's stoma 10a since gravity is failing to assist in directing the bodily fluid towards PRIOR ART bag 1. As such, PRIOR ART bag 1 is only efficient when the PRIOR ART bag 1 is arranged vertically downward from the PRIOR ART skin patch 8 and the patient is provided in a vertical position (i.e., standing or seated upright) to capture most or all of the bodily fluid excreted from the patient's stoma 10a when.

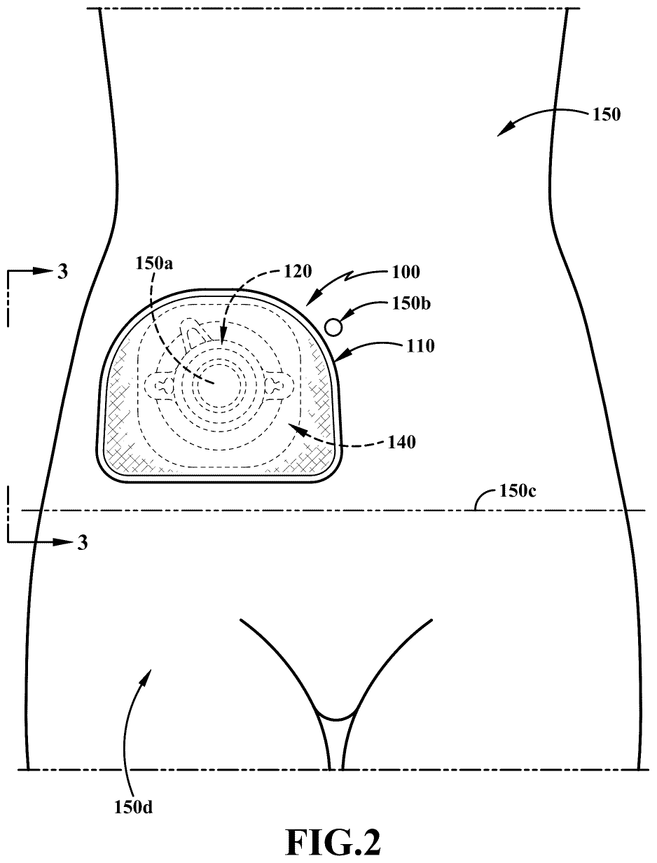

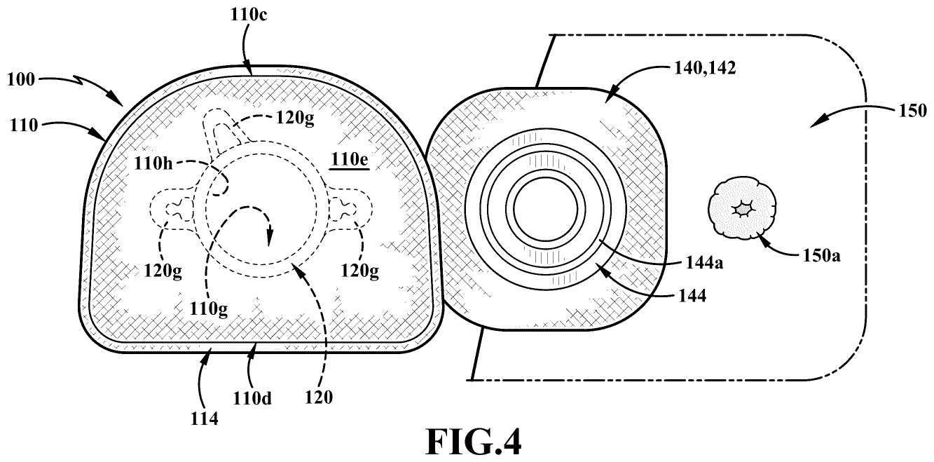

FIGS. 2-11 illustrate a urostomy utility kit or pouch assembly that is configured to provide benefits over the PRIOR ART urostomy bag kit mentioned above and shown in FIGS. 1A-1E. In one instance, urostomy utility kit shown in FIGS. 2-11 includes a urostomy utility pouch that is configured to wick and stabilize bodily fluid excreted from a patient's stoma while also collecting and storing bodily fluid inside of the urostomy utility pouch; such wicking and absorption of bodily fluid from the patient's stoma may occur when the patient is provided in a vertical position, or horizontal position, or other angled positions between vertical and horizontal positions in which gravity may be inefficient in assisting in directing fluid to the urostomy utility pouch. In another instance, urostomy utility kit shown in FIGS. 2-11 is also designed to sit or be positioned above patient's hips and a patient's groin to remove lifestyle nuisances when the patient is wearing the urostomy utility kit. As such, the urostomy utility pouch of urostomy utility kit shown in FIGS. 2-11 is compact and close to the patient for concealment purposes to hide the fact the patient is even wearing the urostomy utility pouch. Such components of urostomy utility kit shown in FIGS. 2-11 are now discussed in greater detail below.

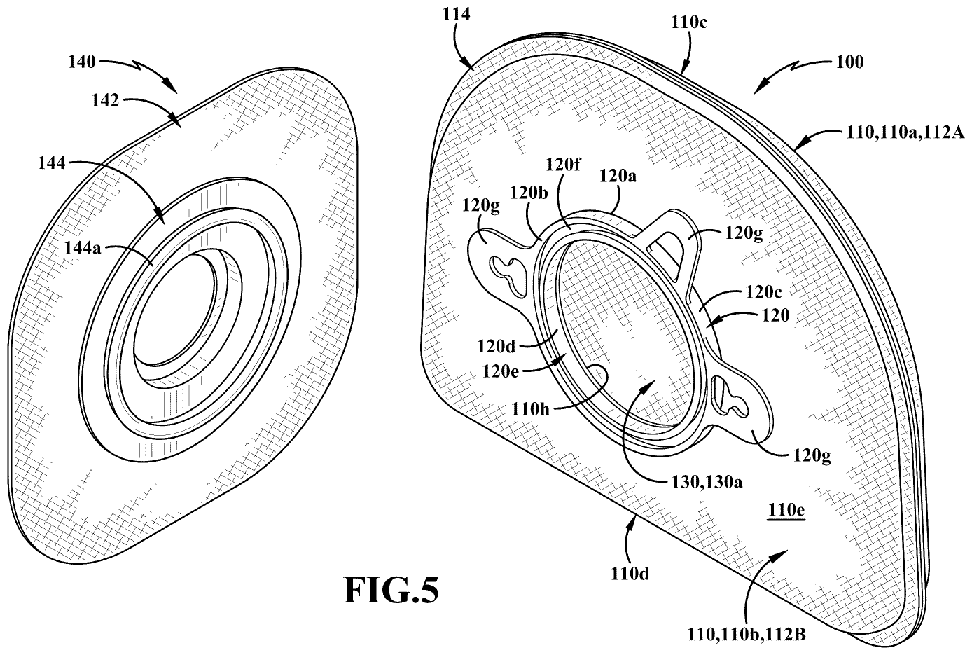

Urostomy utility kit includes a urostomy utility bag or pouch (hereinafter described as “urostomy pouch”) generally referred to as 100 in FIGS. 2-11. As best seen in FIGS. 5-7, urostomy pouch 100 includes a main body 110 having a front or non-engagement end 110a that is configured to face in a first direction or in a direction away from a patient (when used), a rear or engagement end 110b that faces in a second direction opposite to the front end 110a and faces at the patient (when used), and an axis defined therebetween in which the main body 110 expands or contracts along based on the bodily fluid collected and stored inside of main body 110. Main body 110 also includes a top end 110c, a bottom end 110d that is vertically opposite to the top end 110c, and a vertical axis defined therebetween in which the main body 110 expands or contracts along based on the bodily fluid collected and stored inside of main body 110. Main body 110 also includes an exterior surface 110e that extends between front and rear end 110a, 110b and is configured to be in communication with the exterior environment surrounding main body 110, and an interior surface 110f that is opposite to exterior surface 110e and faces inwardly in which the interior surface 110f is free from being in communication with the exterior environment surrounding main body 110.

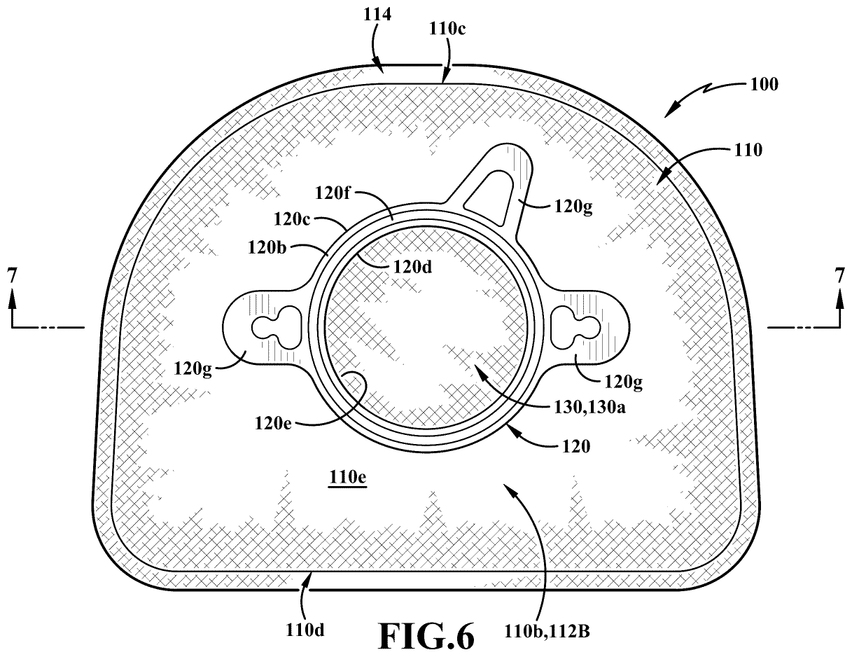

In the present embodiment, main body 110 defines curvilinear shape that begins at the top end 110c and progresses downwardly to the bottom end 110d that is substantially planar or flat when viewed from a rear elevation view (see FIG. 6). In one exemplary embodiment, the curvilinear shape of the main body 110 may be rearranged or reoriented in which the curvilinear shape of main body 110 progresses upward when secured to a patient; in this orientation, the main body 110 is simply rotated 180 degrees from the orientation shown in FIG. 6 in which the rounded or curvilinear shape of the main body 110 is positioned below the substantially planar or flat end of the main body 110. As such, main body 110 may be oriented in at any suitable angle that is comfortable or is ergonomically friendly to the patient when wearing the urostomy pouch 100.

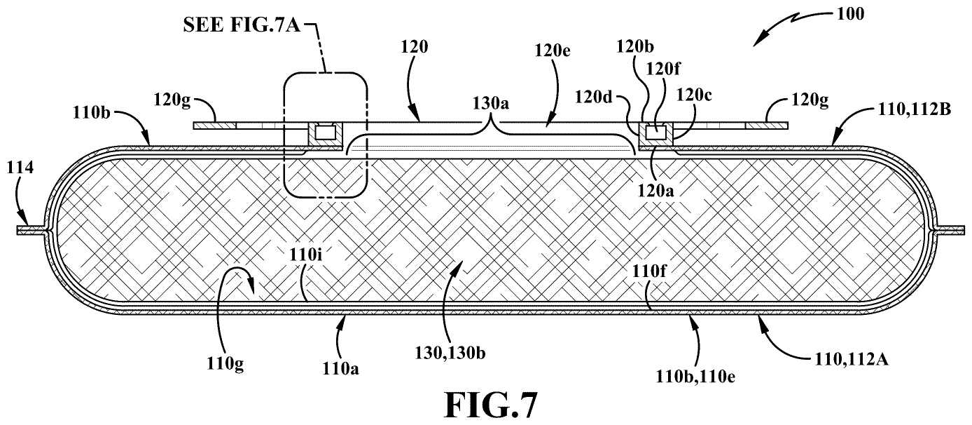

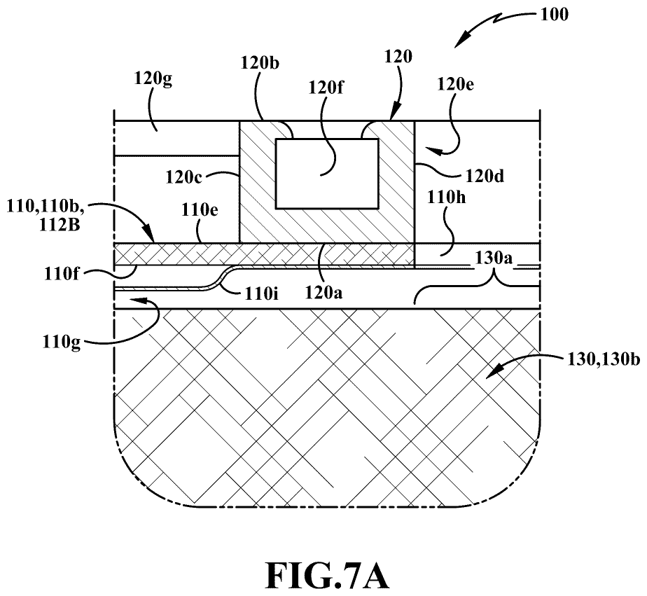

Still referring to main body 110, main body 110 also defines a chamber 110g that is accessible from an opening 110h defined in the rear end 110b of main body 110 (see FIGS. 6-7). In this present embodiment, chamber 110g is configured to collect and store any bodily fluid that is excreted from a patient's stoma and any bodily fluid that avoids being wicked by an absorber of urostomy pouch 100, which is discussed in greater detail below. Main body 110 also includes in an interior liner 110i that is secured to the interior surface 110f of main body 110 inside of the chamber 110g. In this present embodiment, interior liner 110i is configured to prevent bodily fluid or other biohazardous material that is collected by urostomy pouch 100 from leaking or escaping through main body 110 when patient is wearing urostomy pouch 100 or when urostomy pouch 100 is discarded after use. It should be understood that any suitable materials may be used to form interior liner 110i that prevents bodily fluid or other biohazardous material from leaking or escaping through main body 110 while also being biodegradable.

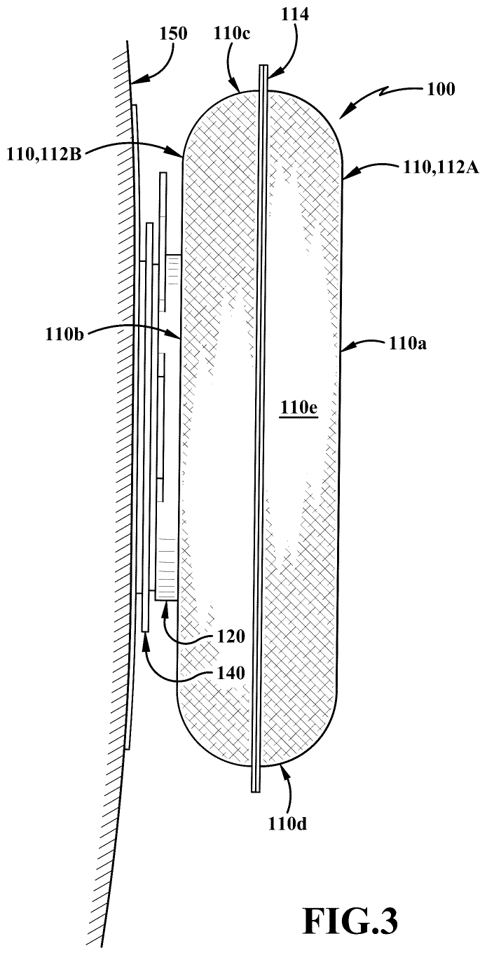

In the present disclosure, main body 110 is also formed by a pair of layers 112A, 112B that is secured together at a peripheral seal 114. As best seen in FIGS. 3, 5, and 7, a first layer 112A of main body 110 that defines the front end 110a of main body 110, and a second layer 112B of main body 110 that defines the rear end 110b of main body 110 and the opening 110h of main body 110. It should also be noted that the pair of layers 112A, 112B also collectively define the top end 110c, the bottom end 110d, the exterior surface 110e, the interior surface 110f, and the chamber 110g while the interior liner 110i is secured to the pair of layers 112A, 112B. It should be noted that while main body 110 is formed by the pair of layers 112A, 112B and held together by peripheral seal 114, any suitable number of layers of material may form main body 110 in other exemplary embodiments. Examples of a number of layers that may form a main body discussed herein include one, two, a plurality, three, four, and other suitable number of layers of material may form the main body dictated by the implementation of the main body.

Urostomy pouch 100 also includes a connector or snap ring 120 that fixedly engages with main body 110. As best seen in FIGS. 5 and 7, connector 120 includes a first engagement end 120a that engages with the main body 110 (particularly the rear end 110b or second layer 112B), a second engagement end 120b that is longitudinally opposite to first engagement end 120a and is spaced apart from main body 110, and a longitudinal axis defined therebetween. Connector 120 also includes an outer cylindrical surface 120c that extends between the first and second engagement ends 120a, 120b and is in communication with the exterior environment surrounding the connector 120, and an inner cylindrical surface 120d that extends between the first and second engagement ends 120a, 120b and faces in an opposite direction to the outer cylindrical surface 120c. Connector 120 also defines a drainage passageway 120e that extends along the inner cylindrical surface 120d between the first and second engagement ends 120a, 120b in which first and second engagement ends 120a, 120b are open ends. It should be noted that drainage passageway 120e is directly aligned with opening 110h of main body 110 so that drainage passageway 120e and chamber 110g of main body 110 are in fluid communication with one another when the urostomy pouch 100 is secured to a patient for collecting and storing bodily fluid.

Still referring to connector 120, connector 120 also includes an interlocking groove 120f. As best seen in FIG. 7A, interlocking groove 120f extends longitudinally into connector 120 from second engagement end 120b towards the first engagement end 120a between outer and inner cylindrical surfaces 120c, 120d. It should be noted that interlocking groove 120f and drainage passageway 120e are free from being in fluid communication with one another along inner cylindrical surface 120d. As discussed in greater detail below, connector 120 is configured to interlockingly couple with a coupler of a skin patch of urostomy utility kit to interlocking engage the urostomy pouch 100 with the skin patch.

Still referring to connector 120, connector 120 also includes at least one pull tab or handle 120g. As best seen in FIGS. 5-6, connector 120 includes at least three pull tabs 120g that radially extend outwardly from the outer cylindrical surface 120c. During use of urostomy utility kit, the patient may grab one or more of the pull tabs 120g of connector 120 to disengage the urostomy pouch 100 from a skin patch of urostomy utility kit when the urostomy pouch 100 meets a filled state and must be discarded; such method of using urostomy utility kit is discussed in greater detail below.

Urostomy pouch 100 also includes an absorber 130 that is operably engaged with the main body 110. As best seen in FIG. 7, absorber 130 is positioned inside of the chamber 110g of main body 110 and interfaces with the interior liner 110i of main body 110. In the present embodiment, a visible or first area 130a of absorber 130 is aligned with the opening 110h of main body 110 and the drainage passageway 120e of connector 120; such visible area 130a of absorber 130 is simply viewable or visible through opening 110h of main body 110 when viewing urostomy pouch 100 from a rear elevation view (see FIG. 6). The visible area 130a is also the initial area or surface of absorber 130 that immediately wicks, absorbs, and stabilizes bodily fluid excreted from a patient's stoma when the urostomy utility kit is being worn by the patient. A second or non-visible area 130b of absorber 130 fills the remaining space of main body 110 inside of chamber 110g that also wicks, absorbs, and stabilizes bodily fluid excreted from a patient's stoma when the urostomy utility kit is being worn by the patient; such non-visible area 130b is simply not viewable or visible through opening 110h of main body 110 when viewing urostomy pouch 100 from a rear elevation view (see FIG. 6).

In the present embodiment, absorber 130 is stored inside of main body 110 while free from being fixed to or secured to interior surface 110f or interior liner 110i as well as free from being fixed to or secured to connector 120. As such, the absorber 130 may move inside of the chamber 110g of main body 110 while still being capable of wicking, absorbing, and stabilizing bodily fluid that is directed into the main body 110. In other exemplary embodiments, other suitable attachment techniques or procedures may be used between main body 110 and absorber 130 and between connector 120 and absorber 130. In one exemplary embodiment, one or more edges or ends of absorber 130 may be fixedly engaged to the interior surface 110f of main body 110 or to the interior liner 110i of main body 110 to prevent unwanted movement or shifting of absorber 130 inside of main body 110. In another exemplary embodiment, one or more edges or ends of absorber 130 may be fixedly engaged to the interior surface 110f of main body 110 inside of the opening 110h. In yet another exemplary embodiment, one or more edges or ends of absorber 130 may be fixedly engaged to the interior surface 110f of main body 110 inside of the opening 110h and fixedly engaged to the inner cylindrical surface 120d of connector 120 inside of drainage passageway 120e.

In the present disclosure, absorber 130 (and other absorbers mentioned herein) is made of a material that is configured to wick and absorb fluid, liquid, or moisture upon initial interaction with the fluid or liquid while being lightweight. It should be understood that any suitable wicking and/or absorbing material that is commercially available may be used to form absorber 130 and other absorbers discussed herein. It should also be understood that such wicking and/or absorbing material used to form absorber 130 also be a biodegradable material if desired.

In the present disclosure, absorber 130 (and other absorbers mentioned herein) is configured to stabilize the bodily fluid inside of the main body 110 to prevent sloshing and/or unwanted movement of such fluid. As such, the absorber 130 is configured to transition and/or turn the bodily fluid or material from a liquid state to a solid-like state to remove sloshing of such bodily fluid when the patient is performing physical activities. The absorber 130 may also wick and stabilize the bodily fluid when the patient is positioned in a vertical orientation, a horizontal orientation, or other suitable orientations or angles due to the capillary action provided by the absorber 130. As such, absorber 130 may wick and stabilize the bodily fluid inside of the main body 110 when gravity fails to provide assistance in directing the bodily fluid into the main body 110 and/or is acting against the pouch 100 such that the bodily fluid would back-out of the main body 110 and back into the patient. Stated differently, the absorber 130 may move the bodily fluid against gravity due to the capillary mechanism that is included in the absorber 130 without the need of being in a desired orientation. Such functionality of urostomy pouch 100 with the inclusion of the absorber 130 provides flexibility to the patient in day-to-day activities.

In the present disclosure, the terms “stabilize”, “stabilized”, “stabilizing”, and other derivatives of “stabilize” commonly used with absorber 130 herein (and other absorbers mentioned herein) may generally refer to act of retaining or maintaining the bodily fluid or material in a solid-like state once the absorber 130 wicks and transitions the bodily fluid from the liquid state to a solid-like or stable state. Once the bodily fluid is “stabilized” by the absorber 130, the bodily fluid is caught and/or trapped in the absorber 130 through capillary actions of said absorber 130 to prevent unwanted movement or sloshing of bodily fluid inside of the main body 110. With such configuration, the bodily fluid becomes part of the absorber 130 and transitions automatically from the liquid state to the solid state. It should be noted that the act of “stabilizing” by the absorber 130 occurs after the absorber 130 initially wicks the bodily fluid and the bodily fluid is excreted from the patient's stoma and enters into the main body 110.

Urostomy utility kit may also include a skin patch 140 that is configured to interlocking engage with the urostomy pouch 100 while also adhering the entire urostomy utility kit to a patient. As best seen in FIG. 5, skin patch 140 includes an adhesive patch 142 that is configured to adhere to a patient's skin; any suitable or commercially available adhesive material or substance may be formed on the adhesive patch 142. Skin patch 140 also includes a coupler 144 that has an attachment clip 144a that interlockingly engages with the connector 120 inside of the interlocking groove 120f. It should be understood that skin patch 140 is similar to PRIOR ART skin patch 8 that is commercially available for urostomy utility kits. Further, as discussed in greater detail below regarding the method of using urostomy pouch 100, skin patch 140 may be a new or existing skin patch that patient may secure urostomy pouch 100 to during use. As such, it should be understood that a step of adhering skin patch 140 may be avoided and/or omitted in certain operations when the skin patch 140 was previously adhered to the patient.

Having now discussed the urostomy utility kit having urostomy pouch 100, a method of using urostomy pouch 100 to collect and store bodily fluid excreted from a patient's stoma is now discussed in greater detail below.

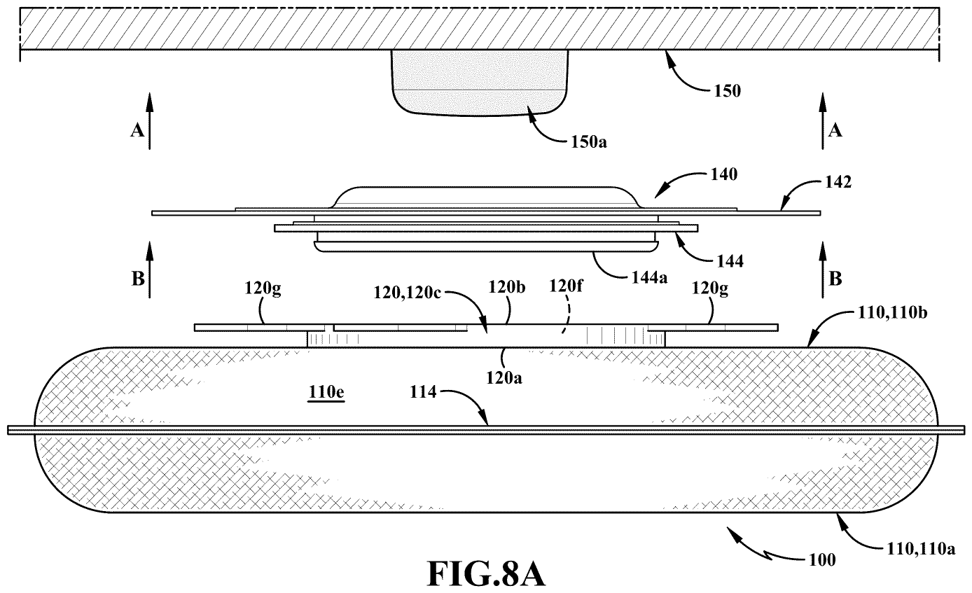

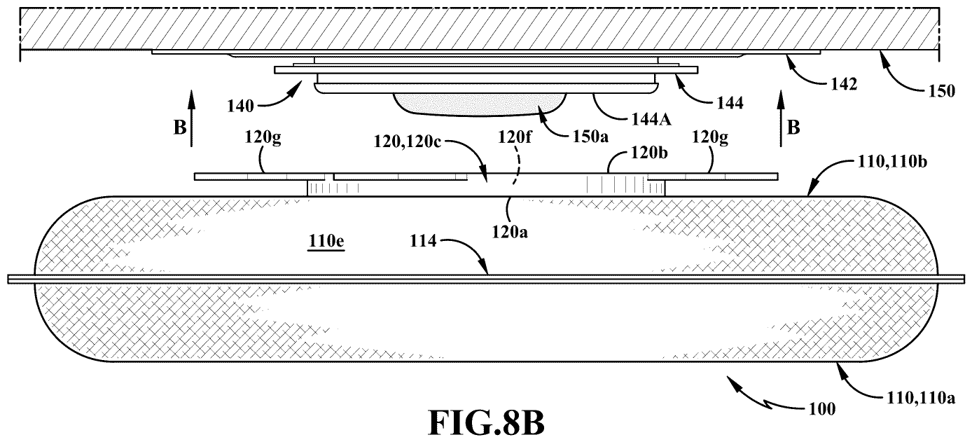

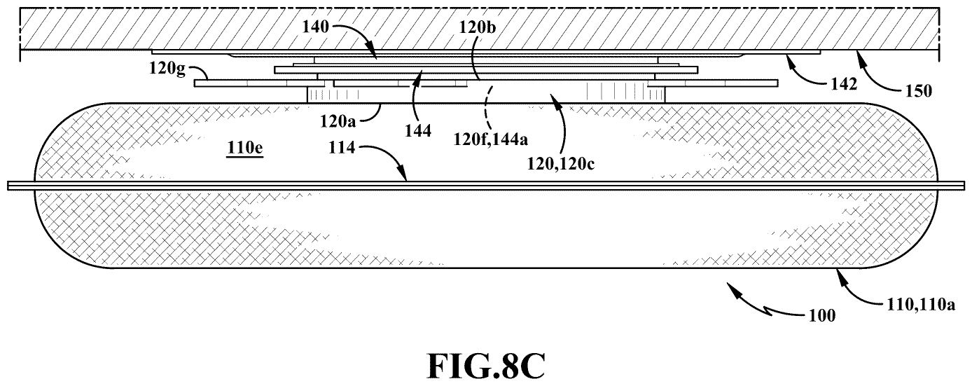

In FIG. 8A, a patient 150 provides both the urostomy pouch 100 and the skin patch 140 near a stoma 150a protruding from patient 150. Initially, patient 150 aligns the skin patch 140 relative to the stoma 150a in which the coupler 144 is aligned with and is intended to surround the stoma 150a. Once aligned, patient 150 may then move the skin patch 140 towards the stoma 150a until the adhesive patch 142 is secured to the patient 150 and the coupler 144 surrounds the stoma 150a; such movement of the skin patch 140 towards the stoma 150a is denoted by arrows labeled “A” in FIG. 8A.

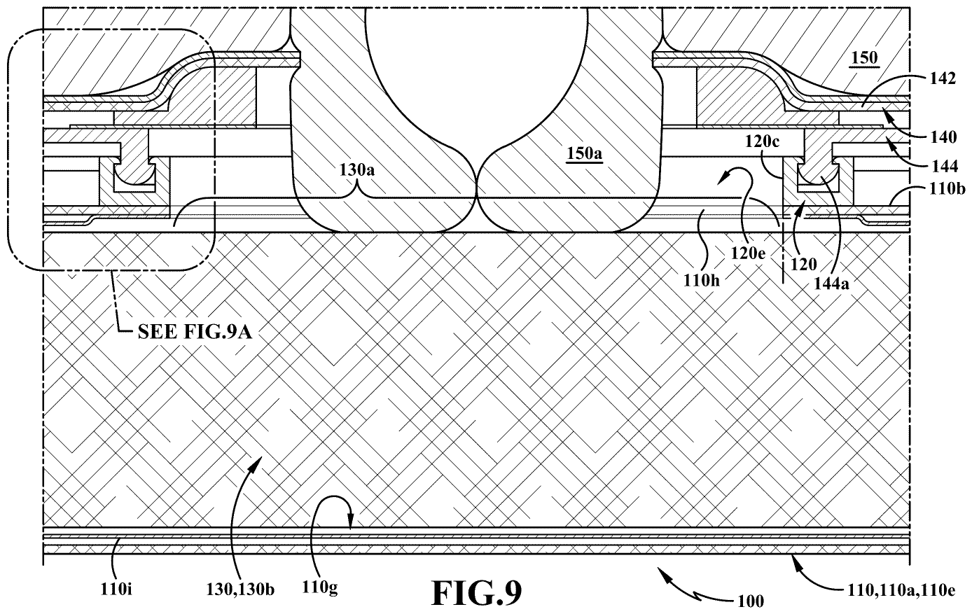

Once skin patch 140 is secured to patient 150, patient 150 may then align the urostomy pouch 100 relative to the skin patch 140 and stoma 150 a (see FIG. 8B). Particularly, patient 150 aligns connector 120 of urostomy pouch 100 with the coupler 144 of skin patch 140 prior to releasably coupling urostomy pouch 100 and skin patch 140 with one another. Once aligned, patient 150 may then move the urostomy pouch 100 towards the skin patch 140 until the attachment clip 144a of skin patch 140 interlockingly engages with the connector 120 inside of interlocking groove 120f; such movement of the urostomy pouch 100 towards the skin patch 140 is denoted by arrows labeled “B” in FIG. 8B. As best seen in FIGS. 9 and 9A, the attachment clip 144a of skin patch 140 interlockingly engages with the connector 120 inside of interlocking groove 120f to suspend the urostomy pouch 100 from the skin patch 140.

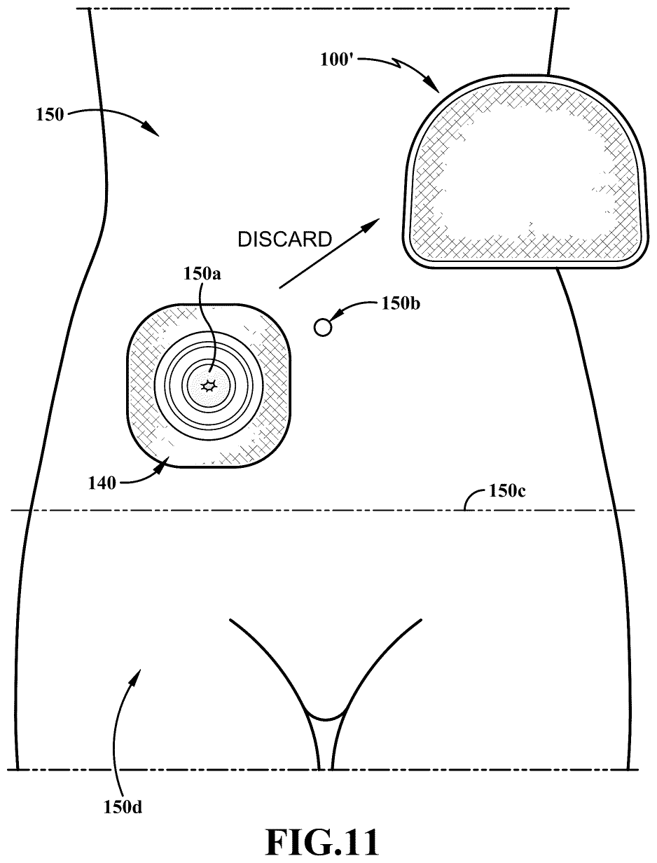

Once urostomy pouch 100 is secured to skin patch 140, urostomy pouch 100 is suspended and/or cantilevered from skin patch 140 that is substantially in line with a patient's umbilicus or navel 150b. Additionally, urostomy pouch 100 is positioned vertically above patient's hips (denoted with a dot-dot-dashed line labeled 150c in FIGS. 2 and 11) and a patient's groin 150d when interlockingly coupled to skin patch 140. In this present disclosure, urostomy pouch 100 is positioned a distance away from the patient's hips 150c and patient's groin 150d which relieves the nuisance and inconvenience from patient as compared to the position of PRIOR ART bag 1 being positioned below patient's hips 10c and proximate to patient's groin 10d.

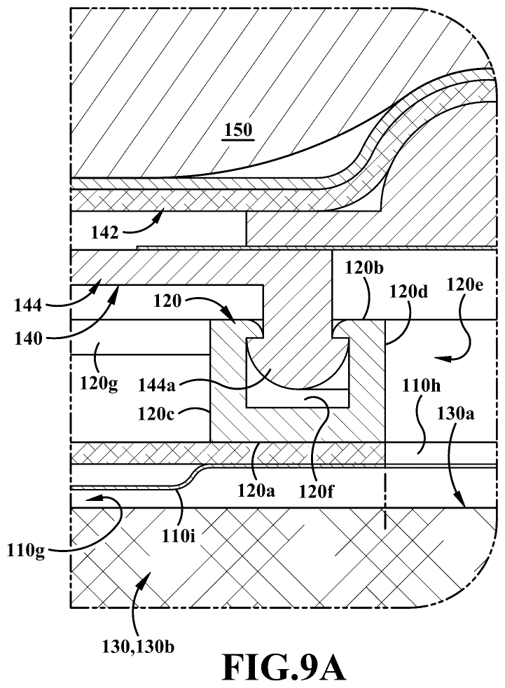

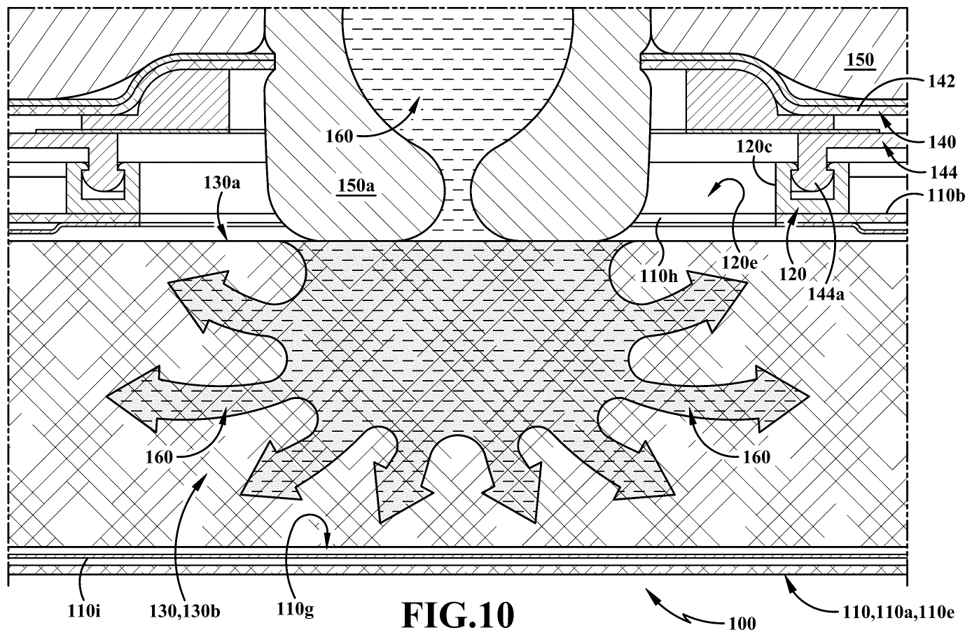

Once urostomy pouch 100 is secured to patient 150, patient's stoma 150a is partially seated inside of skin patch 140 while also passing through skin patch 140 and into urostomy pouch 100. Particularly, and as best seen in FIG. 9, patient's stoma 150a may be housed inside of connector 120, particularly drainage passageway 120e, and extends through the opening 110h of main body 110 into chamber 110g of main body 110. Depending on the arrangement of urostomy pouch 100, absorber 130 may also be directly aligned with patient's stoma 150a and/or directly interface with patient's stoma 150a in order for absorber 130. Such alignment and/or interface between absorber 130 and patient's stoma 150a allows the absorber 130 to be the initial component of urostomy pouch 100 to intercept bodily fluid 160 excreted from patient's stoma 150a before such bodily fluid is collected inside of the chamber 110g (see FIG. 10). Particularly, and as best seen in FIG. 10, the visible area 130a of absorber 130 initially wicks and stabilizes bodily fluid 160 as bodily fluid 160 is excreted from patient's stoma 150a. As the bodily fluid 160 is continuously excreting from patient's stoma 150a, the remaining wicking area of absorber 130 (i.e., non-visible area 130b) assists in wicking and absorbing the bodily fluid 160 as the bodily fluid 160 continues to soak into the absorber 130. Any excessive bodily fluid that is not initially absorbed and wicked by absorber 130 may be collected in the chamber 110g of main body 110.

Such configuration of absorber 130 inside of main body 110 is considered advantageous at least because absorber 130 immediately wicks and stabilizes the bodily fluid 160 without the assistance or need of gravity to direct the bodily fluid 160 into the urostomy pouch 100 as required by PRIOR ART bag 1. As stated previously, urostomy pouch 100 may collect and store bodily fluid 160 while the patient 150 is oriented in a vertical position (e.g., standing or sitting upright) or oriented in a horizontal position (e.g., lying or laying down) due to absorber 130 immediately wicking and absorbing the bodily fluid 160. As such, patient 150 is free to function normally without a concern of reorienting or moving the urostomy pouch 100 depending on the activity the patient 150 is performing due to the use of absorber 130 housed inside of main body 110.

Once the urostomy pouch 100 is full or reaches a desired fill amount (denoted as 100′ in FIG. 11), patient 150 may then disengage or decouple urostomy pouch 100 from skin patch 140. Particularly, patient 150 may decouple urostomy pouch 100 from skin patch 140 by decoupling the attachment clip 144a of skin patch 140 from the interlocking groove 120f of connector 120. If desired, patient 150 may grab and pull on one or more pull tabs 120g of connector 120 to assist in decoupling the connector 120 from the coupler 144 of skin patch 140. Once the urostomy pouch 100 is decoupled from skin patch 140, patient 150 may then simply discard urostomy pouch 100 since urostomy pouch 100 is intended to be a one-use component of urostomy utility kit for sanitary and health concerns; such decoupling and discarding of urostomy pouch 100 is denoted by an arrow labeled “DISCARD” in FIG. 11. It should be noted that during this discarding step, a cap may be coupled to the connector 120 to prevent bodily fluid from escaping; any suitable cap mentioned herein may be used. Once urostomy pouch 100 is discarded, patient 150 may repeat the same steps mentioned above (with or without applying a new skin patch 140) for securing a new urostomy pouch 100 to skin patch 140.

While applying skin patch 140 is being shown as an initial step, skin patch 140 may be an existing or used skin patch that was previously applied and adhered to the patient's skin in other exemplary embodiments.

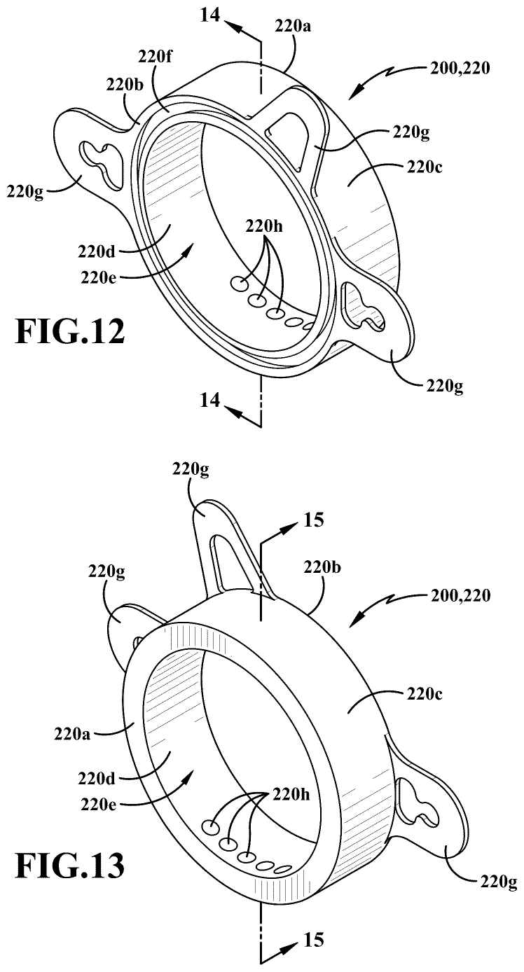

FIGS. 12-17 illustrate another urostomy utility kit that includes a urostomy utility pouch (hereinafter as “urostomy pouch”) 200.

In the present embodiment, urostomy pouch 200 includes a main body 210 that is substantially similar to main body 110 of urostomy pouch 100. Particularly, main body 210 of urostomy pouch 200 includes a front or non-engagement end 210a, a rear or engagement end 210b, a top end 210c, a bottom end 210d, an exterior surface 210e, an interior surface 210f, a chamber 210g, an opening 210h, and an interior liner 210i that are substantially similar to front or non-engagement end 110a, rear or engagement end 110b, top end 110c, bottom end 110d, exterior surface 110e, interior surface 110f, chamber 110g, opening 110h, and interior liner 110i of main body 110 of urostomy pouch 100 discussed above and illustrated in FIGS. 2-11. Main body 210 is also constructed by a pair of layers 212A, 212B fixedly attached at a peripheral seal 214 substantially similar to pair of layers 112A, 112B fixedly attached at peripheral seal 114 of main body 110 discussed above and illustrated in FIGS. 2-11.

In the present embodiment, urostomy pouch 200 also includes a connector 220 that is similar to connector 220 of urostomy pouch 200. Particularly, connector 220 of urostomy pouch 200 includes a first engagement end 220a, a second engagement end 220b, an outer cylindrical surface 220c, an inner cylindrical surface 220d, a drainage passageway 220e, an interlocking groove 220f, and at least one pull tab 220g that are substantially similar to first engagement end 120a, second engagement end 120b, outer cylindrical surface 120c, inner cylindrical surface 120d, drainage passageway 120e, interlocking groove 120f, and at least one pull tab 120g of connector 120 of urostomy pouch 100 discussed above and illustrated in FIGS. 2-11.

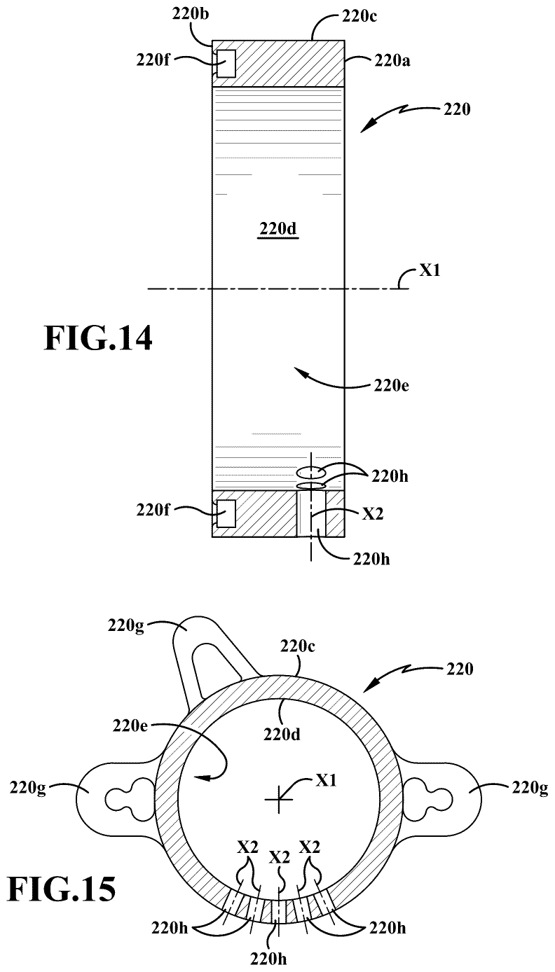

However, in this present embodiment, connector 220 also defines at least one drainage port 220h. As best seen in FIGS. 14-15, connector 220 defines a plurality of drainage ports 220h where each drainage port 220h extends along an axis X2 that is orthogonal to the longitudinal axis X1 of connector 220. Additionally, each drainage port 220h entirely through connector 220 between outer cylindrical surface 220c and inner cylindrical surface 220d; as such, outer cylindrical surface 220c and inner cylindrical surface 220d are in fluid communication with one another at each drainage port of the plurality of drainage ports 220h. Stated differently, each drainage port 220h extends radially through connector 220 between outer cylindrical surface 220c and inner cylindrical surface 220d. In the present embodiment, each drainage port of the plurality of drainage ports 220h is in fluid communication with the drainage passageway 220e defined in connector 220 for drainage operations, which are discussed in greater detail below. Each drainage port of the plurality of drainage ports 220h is, however, free from being in fluid communication with the interlocking groove 220f (see FIG. 14). Moreover, each drainage port of the plurality of drainage ports 220h is positioned between the first and second engagement ends 220a, 220b yet is closer to the first engagement end 220a.

In the present disclosure, the diameter of each drainage port of the plurality of drainage ports 220h is also less than the overall diameter of drainage passageway 220e in the present disclosure. In other exemplary embodiments, diameters of each drainage port of the plurality of drainage ports 220h may vary in size or shape depending on the implementation of connector 220. In one exemplary embodiment, each drainage port of a plurality of drainage ports disclosed herein may be equal to or greater than an overall diameter of a drainage passageway of a connector disclosed herein. In another exemplary embodiment, each drainage port of a plurality of drainage ports disclosed herein may have variable diameters for an intended purpose, including the regulation of bodily fluid draining through each drainage portion.

In the present disclosure, the plurality of drainage ports 220h is defined along a section or portion of the connector 220 and is arranged in a uniform pattern along an linear axis that passes through each drainage port 220h. In other exemplary embodiments, the arrangement and placement of one or more pluralities of drainage ports of a connector discussed herein may be defined along one or more sections or portion of the connector. In one exemplary embodiment, a connector may define at least one plurality set of drainage ports along a first section of the connector and at least another plurality of drainage ports along a second section of the connector that is directly opposite to the first set of drainage ports. In another exemplary embodiment, a connector may define a plurality of drainage ports about the entire connector such that the plurality of drainage ports is continuous along the entire diameter of connector. In yet another exemplary embodiment, a connector may define a drainage slot along a portion of the connector such that the drainage slot is continuous and uninterrupted along said portion of the connector. In yet another exemplary embodiment, a connector may define a plurality of drainage ports along a portion of the connector that is arranged in a non-uniform or random pattern where a linear axis does not pass through each drainage port of the plurality of drainage ports.

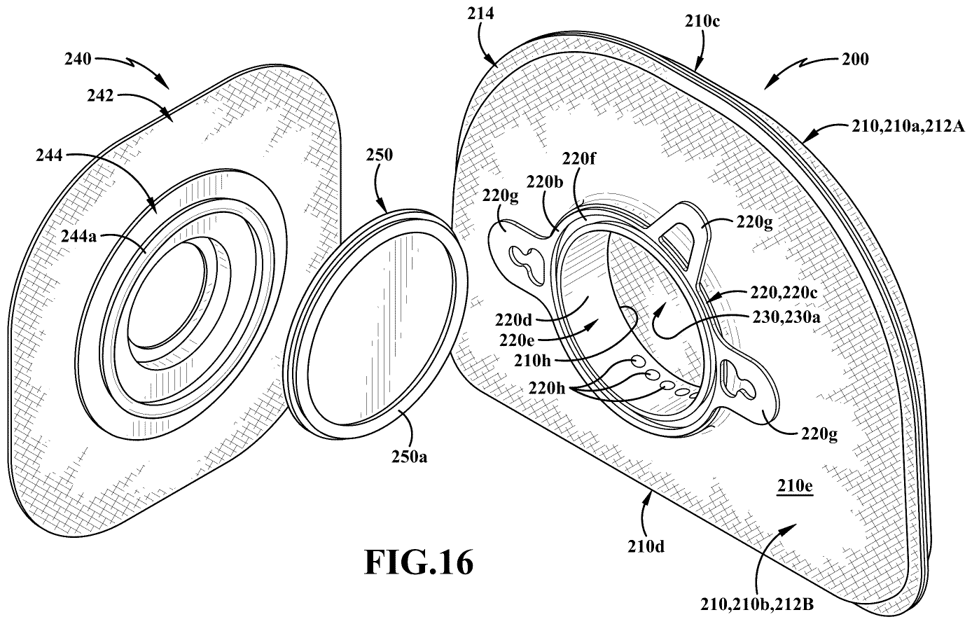

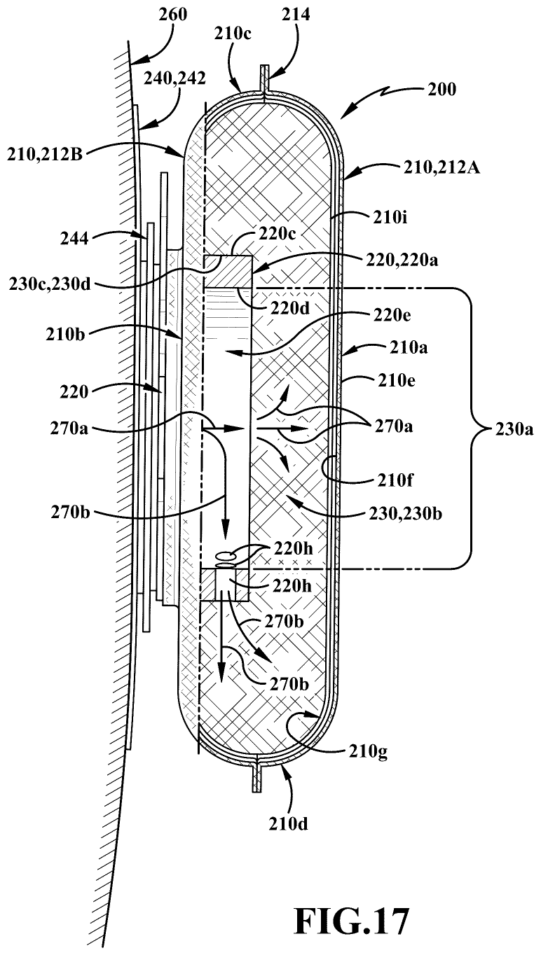

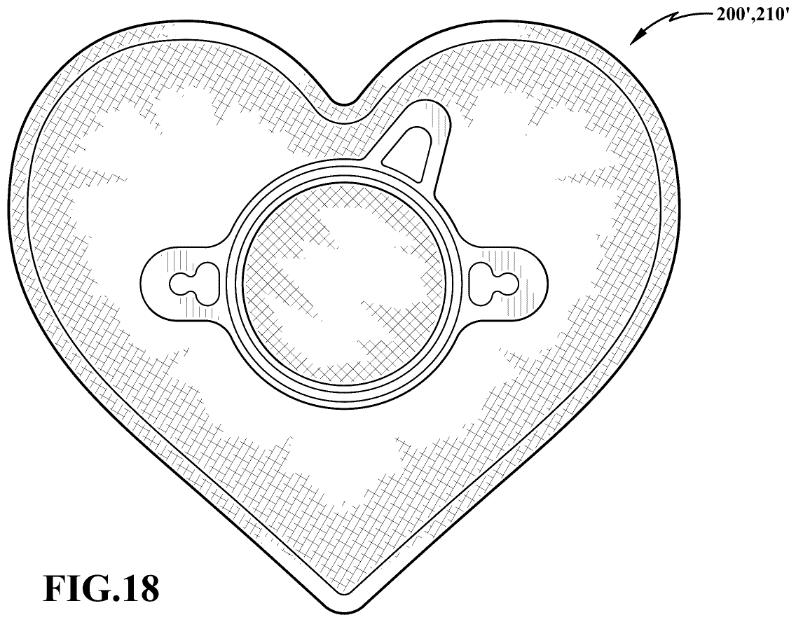

In the present embodiment, urostomy pouch 200 also includes an absorber 230 that is housed inside of the main body 210. As best seen in FIG. 17, absorber 230 includes a first absorbing area or surface 230a that is viewable through opening 210h of main body 210 and is viewable through drainage passageway 220e of connector 220 when viewed from a rear perspective view (see FIG. 16). Still referring to FIG. 17, absorber 230 also includes a second absorbing area 230b that is not viewable through opening 210h of main body 210 nor through drainage passageway 220e of connector 220 when viewed from a rear perspective view. In this embodiment, absorber 230 also defines a counterbore 230c that extends into a peripheral edge of absorber 230 along an lateral surface 230d to a base surface 230e in which a portion of connector 220 is seated inside of counterbore 230c. Particularly, the plurality of drainage ports 220h of connector 220 is seated inside of the counterbore 230c and is in fluid communication with the second absorbing area 230b. It should be noted that counterbore 230c may be any suitable size or shape that is dependent upon the size and shape of the outer profile of connector 220.

In the present disclosure, urostomy pouch 200 may be configured interlockingly couple with and to secure the entire urostomy utility kit to a patient to a skin patch 240. It should be understood that skin patch 240 is substantially similar to skin patch 140 discussed above and includes the same elements of skin patch 140 mentioned above, including adhesive patch 242 and coupler 244 that has an attachment clip 244a substantially similar to adhesive patch 142 and coupler 144 that has attachment clip 144a of skin patch 140.

In the present disclosure, urostomy utility kit also includes a cap 250. As best seen in FIG. 16, cap 250 is configured to interlockingly engage with the connector 220 to prevent bodily fluid from draining or escaping through opening 210h of main body 210 and through drainage passageway 220e of connector 220. Particularly, an attachment clip 250a of cap 250 interlockingly engages with the connector 220 inside of interlocking groove 220f to seal off the drainage passageway 220e when the urostomy pouch 200 is full of bodily fluid excreted from a patient's stoma and is decoupled from the skin patch 240. It should be noted that cap 250 may also be used with other urostomy pouches mentioned herein, including PRIOR ART urostomy bag 1 and urostomy pouch 100, to seal off or temporarily seal off a drainage passageway of a respective connector of urostomy pouch when the urostomy bag or pouch is not in use or when the urostomy bag or pouch is filled with a desired amount of bodily fluid and is decoupled from a skin patch.

Having now discussed the urostomy utility kit having urostomy pouch 200, a method of using urostomy pouch 200 to collect and store bodily fluid excreted from a patient's stoma is now discussed in greater detail below.

For brevity, the steps of installing the urostomy pouch 200 and skin patch 240 around a patient's stoma are identical to the steps of installing the urostomy pouch 100 and skin patch 140 around a patient's stoma; such steps are discussed above and illustrated in FIGS. 8A-10. However, in this present disclosure, connector 220 of urostomy pouch 200 provides more than one drainage direction or pathways for directing and/or draining bodily fluid into at least two absorption areas of absorber 230; such steps are discussed in greater detail below.

As best seen in FIG. 17, connector 220 provides a first drainage pathway 270a that extends through the drainage passageway 220e from the first engagement end 220a to the second engagement end 220b. As such, first drainage pathway 270a is parallel to the longitudinal axis of connector 220 that extends between the first and second engagement ends 220a, 220b of connector 220. In operation, the first drainage pathway 270a flows directly into the first absorbing area 230a of absorber 230 that is outside of the counterbore 230c of absorber 230. The first drainage pathway 270a is also the initial drainage pathway that directs bodily fluid from a patient's stoma into the urostomy pouch 200.

Still referring to FIG. 17, connector 220 also provides a second drainage pathway 270b that extends from the drainage passageway 220e and through each drainage port of the plurality of drainage ports 220h. As such, the second drainage pathway 270b defined through each drainage port of the plurality of drainage ports 220h is substantially orthogonal to the longitudinal axis of connector 220 that extends between the first and second engagement ends 220a, 220b of connector 220 and to the first drainage pathway 270a. In operation, the second drainage pathways 270b flows directly into the second absorbing area 230b of absorber 230 that is outside of the counterbore 230c of absorber 230. In most operations, the second drainage pathway 270b is the secondary drainage pathway that directs bodily fluid from a patient's stoma into the urostomy pouch 200. During certain drainage operations, one or both of the first and second drainage pathways 270a, 270b may be utilized depending on the volume or flow of bodily fluid that excretes from patient's stoma.

Such use of two drainage pathways 270a, 270b is considered advantageous at least because the absorber 230 may wick and absorb bodily fluid at more than one location. With this configuration, the absorber 230 may less likely be fully saturated with bodily fluid at one location due to the connector 220 being able to direct at least two streams of bodily fluid to the absorber 230. Additionally, the two pathways 270a, 270b may also be advantageous in directing bodily fluid to the absorber 230 depending on position of the patient (either stationed in a vertical position or a horizontal position) so that the bodily fluid does not leak or escape from the urostomy pouch 200 or skin patch 240 as the bodily fluid transitions through the urostomy utility kit. In one example, one or more drainage ports 220h may be primarily used to drain bodily fluid along the second drainage pathways 270b due to the patient lying in the horizontal position or is seated at an angle between the vertical position and the horizontal position.

As discussed previously, main bodies 110, 210 define curvilinear shapes that begins at the top ends 110c, 210c and progress downwardly to the bottom ends 110d, 210d that is substantially planar or flat when viewed from a rear elevation view of main body 110 (see FIG. 6) or a rear perspective view of main body 210 (see FIG. 16). In other exemplary embodiments, a main body of a urostomy pouch disclosed herein may define other suitable shapes depending on the implementation of urostomy pouch. In one example, and as best seen in FIG. 17, an alternative main body 210′ of alternative urostomy pouch 200′ defines a heart-shaped configuration.

Unless explicitly stated that a particular shape or configuration of a component is mandatory, any of the elements, components, or structures discussed herein may take the form of any shape. Thus, although the figures depict the various elements, components, or structures of the present disclosure according to one or more exemplary embodiments, it is to be understood that any other geometric configuration of that element, component, or structure is entirely possible. For example, instead of the main bodies 110, 210, 210′ being curvilinear-shaped or heart-shaped, a main body of a urostomy pouch disclosed herein may be semi-circular triangular, rectangular or square, pentagonal, hexagonal, heptagonal, octagonal, decagonal, dodecagonal, diamond shaped or another parallelogram, trapezoidal, star-shaped, oval, ovoid, lines or lined, teardrop-shaped, cross-shaped, donut-shaped, heart-shaped, arrow-shaped, crescent-shaped, any letter shape, or any other type of regular or irregular, symmetrical or asymmetrical configuration.

As provided herein, any urostomy pouches disclosed herein, including urostomy pouches 100, 200, 200′, may be formed from any suitable materials that are commercially available. In one exemplary embodiment, urostomy pouches disclosed herein, including urostomy pouches 100, 200, 200′, may be formed from a biodegradable material while being capable of retaining the bodily fluid after being discarded.

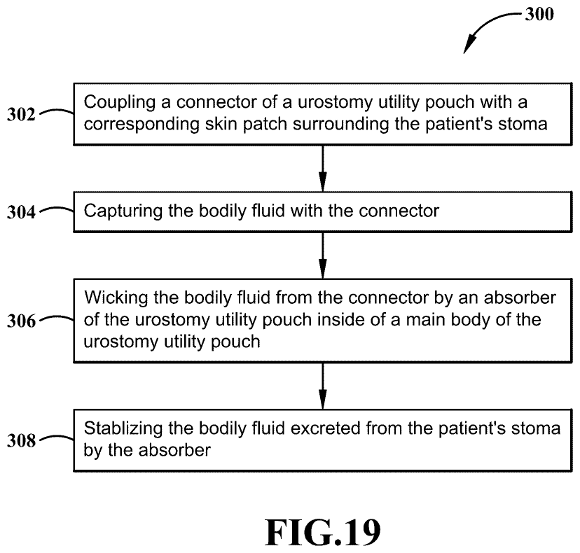

FIG. 19 illustrates a method 300 of storing bodily fluid excreted from a patient's stoma. An initial step 302 of method 300 includes coupling a connector of a urostomy utility pouch with a corresponding skin patch surrounding the patient's stoma. Another step 304 of method 300 includes capturing the bodily fluid with the connector. Another step 306 of method 300 includes wicking the bodily fluid from the connector by an absorber of the urostomy utility pouch inside of a main body of the urostomy utility pouch. Another step 308 of method 300 includes storing the bodily fluid excreted from the patient's stoma by the absorber.

In other exemplary embodiments, method 300 may include optional steps of further limiting steps of storing bodily fluid excreted from a patient's stoma. In one exemplary embodiment, method 300 may further include a step of directing the bodily fluid towards an opening defined the urostomy utility pouch and into a chamber of the urostomy utility pouch. In another exemplary embodiment, method 300 may further include a step of positioning the urostomy utility pouch entirely above patient's hips and remote from a patient's groin. In another exemplary embodiment, method 300 may further include that the steps of directing the bodily fluid and wicking the bodily fluid further comprises: directing the bodily fluid into the chamber and the absorber through a drainage passageway defined in the connector; and wicking the bodily fluid from the connector by the absorber at a first absorbing area. In another exemplary embodiment, method 300 may further include that the steps of directing the bodily fluid and wicking the bodily fluid further comprises: directing the bodily fluid from the drainage passageway and into the chamber and the absorber through at least one drainage port defined in the connector; and wicking the bodily fluid from the connector by the absorber at a second absorbing area that is substantially orthogonal to the first absorbing area of the absorber. In another exemplary embodiment, method 300 may further include that the step of coupling the connector of the urostomy utility pouch with the corresponding skin patch further comprises: engaging an attachment clip of the skin patch with an interlocking groove defined in the connector; and wherein the steps of directing the bodily fluid and wicking the bodily fluid further comprises: directing the bodily fluid into the chamber and the absorber through at least one drainage port defined in the connector; wherein the interlocking groove and the at least one drainage port are free from being in fluid communication with one another. In another exemplary embodiment, method 300 may further include steps of removing the urostomy utility pouch from the skin patch; engaging a cap with the connector; and sealing the chamber of the urostomy utility pouch with the cap at the opening. In another exemplary embodiment, method 300 may further include a main body of the urostomy utility pouch is made of biodegradable material.

As described herein, aspects of the present disclosure may include one or more mechanical or other similar secondary components and/or systems therein. The present disclosure is therefore contemplated and will be understood to include any necessary operational components thereof. It will be further understood that any connections between various components not explicitly described herein may be made through any suitable means including mechanical fasteners, or more permanent attachment means, such as welding or the like. Alternatively, where feasible and/or desirable, various components of the present disclosure may be integrally formed as a single unit.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any flowchart and/or block diagrams in the Figures illustrate some exemplary architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

For example, although the urostomy pouch of the present disclosure is described as a complete unit within the present disclosure, it is to be understood that some of the components or features detailed herein can be supplied as a retrofit kit. This approach enables the provision of only certain parts necessary to upgrade a legacy device to the specifications of urostomy pouch of the present disclosure. Essentially, instead of requiring the replacement of the entire device, the retrofit kit allows for the selective enhancement of specific components. This could allow a user or operator to efficiently upgrade its/their existing legacy devices, systems, or assemblies to achieve the performance and functionality of the urostomy pouch of the present disclosure without a full replacement. In the event that a component or portion of the urostomy pouch of the present disclosure is provided as part of a retrofit kit, those components may be integrated into legacy devices, systems or assemblies to upgrade the same. By facilitating partial upgrades, it addresses the need for continuous improvement and adaptation in dynamic environments where complete replacement might be neither feasible nor necessary. As a result, a user or operator would be able to make an enhancement, thereby extending the lifecycle, optimizing, or improving those legacy devices, systems, or assemblies.

The articles “a” and “an,” as used herein in the specification and in the claims, unless clearly indicated to the contrary, should be understood to mean “at least one.” The phrase “and/or” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. As another example, “at least one of: A, B, or B” is intended to cover A, B, C, A-B, A-C, B-C, and A-B-C, as well as any combination with multiple of the same item.

While components of the present disclosure are described herein in relation to each other, it is possible for one of the components disclosed herein to include inventive subject matter, if claimed alone or used alone. In keeping with the above example, if the disclosed embodiments teach the features of A and B, then there may be inventive subject matter in the combination of A and B, A alone, or B alone, unless otherwise stated herein.

As used herein in the specification and in the claims, the term “effecting” or a phrase or claim element beginning with the term “effecting” should be understood to mean to cause something to happen or to bring something about. For example, effecting an event to occur may be caused by actions of a first party even though a second party actually performed the event or had the event occur to the second party. Stated otherwise, effecting refers to one party giving another party the tools, objects, or resources to cause an event to occur. Thus, in this example a claim element of “effecting an event to occur” would mean that a first party is giving a second party the tools or resources needed for the second party to perform the event, however the affirmative single action is the responsibility of the first party to provide the tools or resources to cause said event to occur.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present disclosure.

An embodiment is an implementation or example of the present disclosure. Reference in the specification to “an embodiment,” “one embodiment,” “some embodiments,” “one particular embodiment,” “an exemplary embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” “an exemplary embodiment,” or “other embodiments,” or the like, are not necessarily all referring to the same embodiments. Furthermore, the use of any and all examples or exemplary language (“e.g.,” “such as,” or the like) is intended merely to better illustrate or illuminate the embodiments and does not pose a limitation on the scope of that or those embodiments. No language in this specification should be construed as indicating any unclaimed element as essential to the practice of the disclosed embodiment.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element or “another” element, that does not preclude there being more than one of the additional element or another element.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein. Further, recitation of ranges of values herein are not intended to be limiting, referring instead individually to any and all values falling within that range, unless otherwise indicated herein, and each separate value within such range is incorporated into the specification as if it were individually recited herein.

Additionally, the method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

To the extent that the present disclosure has utilized the term “invention” in various titles or sections of this specification, or in the context of those sections, this term has been included as required by the formatting requirements of word document submissions (i.e., docx submissions) pursuant the guidelines/requirements of the United States Patent and Trademark Office and shall not, in any manner, be considered a disavowal of any subject matter.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of various embodiments of the disclosure are examples and the disclosure is not limited to the exact details shown or described.

What is claimed is:

1. A urostomy utility pouch, comprising:
a main body having an exterior surface exposed to a surrounding environment and an interior surface opposite to the exterior surface and defining a chamber;
an opening defined in the main body providing access to the chamber;
a connector operably engaged with the main body inside of the opening and adapted to couple with a skin patch positioned about a patient's stoma; and
an absorber operably engaged with the interior surface of the main body inside of the chamber;
wherein the absorber is configured to wick and stabilize bodily fluid from the patient's stoma while the main body is configured to hold the bodily fluid inside of the chamber.

2. The urostomy utility pouch of claim 1, wherein the pouch is configured to be positioned entirely above patient's hips and remote from a patient's groin.

3. The urostomy utility pouch of claim 1, wherein the connector comprises:
an engagement end adapted to engage with an attachment clip of the skin patch;
a chamber end opposite to the engagement end and positioned inside of the chamber; and
a drainage passageway defined between the engagement end and the chamber end that is configured to collect and guide the bodily fluid from the patient's stoma to a first absorbing area of the absorber and the chamber.

4. The urostomy utility pouch of claim 3, wherein the absorber engages with the chamber end and is positioned outside of the drainage passageway.

5. The urostomy utility pouch of claim 3, wherein the connector further comprises:
at least one drainage port extending between an inner surface of the connector and an outer surface of the connector and being in fluid communication with the drainage passageway;
wherein the at least one drainage port is configured to drain the bodily fluid from the drainage passageway to a second absorbing area of the absorber and the chamber.

6. The urostomy utility pouch of claim 5, further comprising:
a first drainage pathway defined along a length of the drainage passageway of the connector; and
a second drainage pathway defined along a length of the at least one drainage port of the connector that is non-parallel to the first drainage pathway.

7. The urostomy utility pouch of claim 5, wherein the connector further comprises:
an interlocking groove extending from the engagement end to a base wall of the connector;
wherein the interlocking groove is adapted to interlockingly engage with an attachment clip of the skin patch.

8. The urostomy utility pouch of claim 7, wherein the at least one drainage port and the interlocking groove are free from being in fluid communication with one another along the inner surface of the connector.

9. The urostomy utility pouch of claim 3, wherein the connector further comprises:
a plurality of drainage ports extending between an inner surface of the connector and an outer surface of the connector and being in fluid communication with the drainage passageway;
wherein each drainage port of the plurality of drainage ports is configured to drain the bodily fluid from the drainage passageway to the chamber.

10. The urostomy utility pouch of claim 1, further comprising:
cap removably engageable with the connector to prevent the bodily fluid from escaping the chamber;
wherein the cap is engaged with the connector when the connector is decoupled from the skin patch.

11. The urostomy utility pouch of claim 1, wherein the connector further comprises:
at least one pull tab operably engaged with an outer surface of the connector.

12. The urostomy utility pouch of claim 1, wherein the main body is made of biodegradable material.

13. A method of storing bodily fluid excreted from a patient's stoma, comprising steps of
coupling a connector of a urostomy utility pouch with a corresponding skin patch surrounding the patient's stoma;
capturing the bodily fluid with the connector;
wicking the bodily fluid from the connector by an absorber of the urostomy utility pouch inside of a main body of the urostomy utility pouch; and
stabilizing the bodily fluid excreted from the patient's stoma by the absorber.

14. The method of claim 13, further comprising:
positioning the urostomy utility pouch entirely above patient's hips and remote from a patient's groin.

15. The method of claim 13, further comprising:
directing the bodily fluid into a chamber of the main body and the absorber through a drainage passageway defined in the connector; and
wicking the bodily fluid from the connector by the absorber at a first absorbing area.

16. The method of claim 15, further comprising:
directing the bodily fluid from the drainage passageway and into the chamber and the absorber through at least one drainage port defined in the connector; and
wicking the bodily fluid from the connector by the absorber at a second absorbing area that is substantially orthogonal to the first absorbing area of the absorber.

17. The method of claim 13, wherein the step of coupling the connector of the urostomy utility pouch with the corresponding skin patch further comprises:
engaging an attachment clip of the skin patch with an interlocking groove defined in the connector; and
wherein the steps of directing the bodily fluid and wicking the bodily fluid further comprises:
directing the bodily fluid into the chamber and the absorber through at least one drainage port defined in the connector;
wherein the interlocking groove and the at least one drainage port are free from being in fluid communication with one another.

18. The method of claim 13, further comprising:
removing the urostomy utility pouch from the skin patch;
engaging a cap with the connector; and
sealing the chamber of the urostomy utility pouch with the cap at the opening.

19. The method of claim 13, wherein a main body of the urostomy utility pouch is made of biodegradable material.

20. A urostomy utility kit, comprising:
a urostomy utility pouch removably coupled with a skin patch surrounding a patient's stoma, the urostomy utility pouch comprising:
a main body having an exterior surface exposed to a surrounding environment and an interior surface opposite to the exterior surface and defining a chamber;
an opening defined in the main body providing access to the chamber;
a connector operably engaged with the main body inside of the opening and adapted to couple with the skin patch positioned about the patient's stoma; and
an absorber operably engaged with the interior surface of the main body inside of the chamber;
wherein the absorber is configured to wick bodily fluid from the patient's stoma while the main body is configured to hold the bodily fluid inside of the chamber; and
a cap removably engageable with the urostomy utility pouch to prevent the bodily fluid from escaping the chamber.
